(19) United States
(12) Patent Application Publication
(10) Pub. No.: US 2009/0142838 A1
(43) Pub. Date: Jun. 4, 2009

(54) Methods for expressing rnp particles in eukaryotic cells

(76) Inventors: Xiaoxia Cui, Saint Louis, MO (US); Alan Lambowitz, Austin, TX (US); Roland Saldanha, Austin, TX (US)

Correspondence Address: CALFEE HALTER & GRISWOLD, LLP, 800 SUPERIOR AVENUE, SUITE 1400, CLEVELAND, OH, 44114, US

(21) Appl. No.: 11/629,441

(22) PCT Filed: Jun. 14, 2005
(86) PCT No.: PCT/US05/20893

§ 371 Date: Jun. 29, 2007

Related U.S. Application Data
(60) Provisional application No. 60/579,212, filed on Jun. 14, 2004.

(51) Int. Cl. C12N 15/87; C12N 15/00

(52) U.S. Cl. 435/455; 435/320.1

(57) ABSTRACT

Provided herein are nucleic acid constructs and methods for producing or enhancing the production of group II intron RNP particles in eukaryotic cells. The present methods comprise introducing at least one nucleic acid construct comprising a nucleic acid encoding a modified or wild type group II intron RNA and a wild-type or modified group II intron-encoded protein into the eukaryotic cell, and maintaining the cell under conditions that allow for expression of the group II intron RNA and the group II intron-encoded protein in the cell. The nucleic acid encoding the group II intron RNA is operably linked to an RNA polymerase I, an RNA polymerase II, or an RNA polymerase III promoter, and the nucleic acid encoding the group II intron-encoded protein is operably linked to an RNA polymerase II promoter. In certain embodiments, a subcellular localization signal is attached to the group II intron-encoded protein.

### CROSS REFERENCE TO RELATED APPLICATIONS

This application claims priority to U.S. Provisional Application No. 60/579,212 filed Jun. 14, 2004, which is incorporated herein by reference in its entirety.

### STATEMENT OF GOVERNMENT SUPPORT

This work was supported, at least in part, by grant number GM37949 from the Department of Health and Human Services, National Institutes of Health. The United States government has certain rights in this invention.

### BACKGROUND

The present invention relates to methods of enhancing the expression in eukaryotic cells of RNP particles that are capable of catalyzing the cleavage of single-stranded and double-stranded DNA substrates at specific recognition or target sites, and of concomitantly inserting nucleic acid molecules into the DNA substrate at the target site. Such RNP particles are useful tools, particularly for genome mapping and for genetic engineering. Structurally, the ribonucleoprotein (RNP) particles of the present invention comprise an excised, group II intron RNA and a group II intron-encoded protein, which is bound to the group II intron RNA. Group II intron RNP particles, as described herein, are useful analytical tools for determining the presence and location of a particular target sequence in a cellular DNA substrate. Group II intron RNP particles as described herein are also useful tools for rendering certain genes within the eukaryotic cell's genomic DNA nonfunctional. Group II intron RNP particles, as described herein, are also useful tools for inserting a nucleic acid into the cleavage site, thus changing the characteristics of the cellular DNA and RNA and protein molecules encoded by the cellular DNA. Accordingly, constructs and methods which can be used to enhance the production of group II intron RNP particles in eukaryotic cells are desirable.

### SUMMARY OF THE INVENTION

The present application provides nucleic acid constructs and methods for producing or enhancing the production of group II intron RNP particles in eukaryotic cells. The group II RNP particles comprise a wild-type or, preferably, a modified group II intron RNA associated with a wild type or modified group II intron-encoded protein. The group II intron RNA is targeted to interact with a DNA substrate in the eukaryotic cell. The group II intron RNP particles of the present invention are capable of catalyzing the cleavage, at a specific target site, of single-stranded and double-stranded DNA substrates that are present in eukaryotic cells, including genomic DNA substrates, and introducing a heterologous nucleic acid into the target site.

The present methods comprise introducing at least one nucleic acid construct comprising a nucleic acid encoding a modified or wild type group II intron RNA and a wild-type or modified group II intron-encoded protein into the eukaryotic cell, and maintaining the cell under conditions that allow for expression of the group II intron RNA and the group II intron-encoded protein in the cell. The nucleic acid encoding the group II intron RNA is operably linked to an RNA polymerase I, an RNA polymerase II, or an RNA polymerase III promoter, and the nucleic acid encoding the group II intron-encoded protein is operably linked to an RNA polymerase II promoter. In certain embodiments, the group II intron RNA and group II intron-encoded protein are encoded by the same nucleic acid segment, i.e., the open reading frame for the group II intron-encoded protein is located in domain IV of the nucleic acid molecule encoding the group II intron RNA. In this embodiment, both the group II intron RNA and the group II intron encoded protein are operably linked to the same promoter, preferably an RNA polymerase II promoter. In other preferred embodiments the group II intron RNA lacks an open reading frame encoding the protein and the protein is encoded by a different nucleic acid in the construct or is encoded by a separate construct. In either case, the nucleic acid encoding the group II intron RNA may be operably linked to a first promoter, e.g., an RNA polymerase I promoter, and the nucleic acid encoding the group II intron-encoded protein may be operably linked to a second promoter, e.g., an RNA polymerase II promoter. In those cases where the DNA encoding the group II intron RNA and the group II intron-encoded protein are operably linked to the same promoter, e.g., an RNA polymerase II promoter, and the sequence encoding the protein is downstream of the sequence encoding the group II intron RNA, the construct preferably also comprises an internal ribosome entry site (IRES) between the sequence encoding the group II intron RNA and the sequence encoding the protein. In certain embodiments, the construct further comprise a nuclear, nucleolar, or other subcellular localization signal encoding sequence operably linked to the sequence encoding the group II intron-encoded protein.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended Claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE FIGURES

FIG. 1. Resynthesis of the LtrA gene using codons preferred by human cells. The LtrA sequence is represented by the thick, horizontal line, on which vertical bars mark the boundaries of six segments divided for cloning purposes. Horizontal bars represent overlapping synthetic DNA oligonucleotides used as templates in PCR reactions to amplify each segment. Arrowheads show smaller primers used to amplify each segment. Letters next to arrowheads represent restriction sites that were later used in cloning. R: EcoRI; H: HindIII; X: XbaI. Grey arrows represent the two complementary oligonucleotides used to amplify segments 5 and 6 into one piece. Each segment was flanked by two restriction sites that are unique within the fragment and were later used to ligate all the pieces together.

FIG. 2 Expression constructs of LtrA and intron RNA. (a) LtrA expression vector phLtrA. Pcmv refers to the human cytomegalovirus (CMV) immediate early promoter; IVS stands for intervening sequence, i.e., a nuclear intron efficiently spliced by spliceosomes; hLtrA is the human codon-optimized LtrA open reading frame; NLS is the SV40 nuclear localization signal; and pA represents polyA signal. (b) Intron expression vector pHHWT. PpolI refers to human RNA polymerase I promoter; intron represents the lactococcal L1.LtrB intron with the majority of the LtrA ORF deleted; T stands for the pol I terminator. Constructs with a Pol II or Pol III promoter have a similar configuration.

FIG. 3 The expression of LtrA and splicing of the intron RNA. (a) Western analysis of lysates of cells transiently or stably expressing hLtrA. Lane 1, LtrA purified from E. coli as a positive control; lane 2, untransfected 293 cells; lane 3, 293 cells transfected with pCMV/nuc/myc vector; lane 4, 293 cells transfected with a vector containing non-optimized LtrA ORF; lane 5, 293 cells transfected with pLtrA (see FIG. 2a); lanes 6 and 7, two stable cell lines of 293 expressing hLtrA. (b) RT-PCR of total RNAs from 293 cells transfected with pHHWT (lane 1), or from the two hLtrA stable cell lines transfected with pHHWT (lanes 2 and 5), a vector with intron driven by a CMV promoter (lanes 3 and 6), a vector with intron under the U6 promoter (a Pol III promoter) (lanes 4 and 7). Positions of precursor and ligated exons are labeled on the right.

FIG. 4. Localization of LtrA. The left panels are immunofluorescence analysis on transiently transfected 293 cells (a), COS-7 cells (b and c), and stable hLtrA cell line #25 (d), using an anti-LtrA antibody and FITC-conjugated secondary antibody. The middle panels are nuclear staining of the same sets of cells. The right panels are superimpositions of the first two, showing whether or not the LtrA protein is localized in the nucleus.

FIG. 5 shows the L1.LtrB intron DNA sequence and portions of the nucleotide sequence of the flanking exons E1 and E2, SEQ.ID.NO.5, and the nucleotide sequence of the open reading frame, of the L1.LtrB intron intron SEQ. ID. NO. 6.

FIG. 6. Group II intron RNA splicing mechanism and secondary structure. A. Splicing occurs via two sequential transesterification reactions. In the first, nucleophilic attack at the 5′-splice site by the 2′ OH of a bulged A-residue in domain VI results in cleavage of the 5′-splice site coupled to formation of lariat intermediate. In the second, nucleophilic attack at the 3′-splice site by the 3′ OH of the cleaved 5′ exon results in exon ligation and release of the intron lariat. B. The conserved secondary structure consists of six double-helical domains (DI-DVI) emanating from a central wheel, with subdomains indicated by lower case letters (e.g., DIVa). The ORF is encoded within DIV (dotted loop), and DIVa is the high-affinity binding site for the intron-encoded protein (IEP). Greek letters indicate sequences involved in tertiary interactions. EBS and IBS refer to exon- and intron-binding sites, respectively. As used herein, the term “EBS” also refers to hybridizing sequences in the intron RNA that base pair with recognition sites or sequences in a DNA substrate in the eukaryotic cell. Some key differences between subgroup IIA, IIB, and IIC introns are indicated within dashed boxes, but additional smaller differences are not shown.

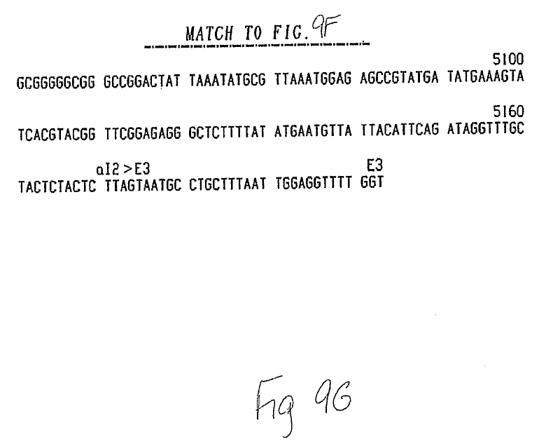

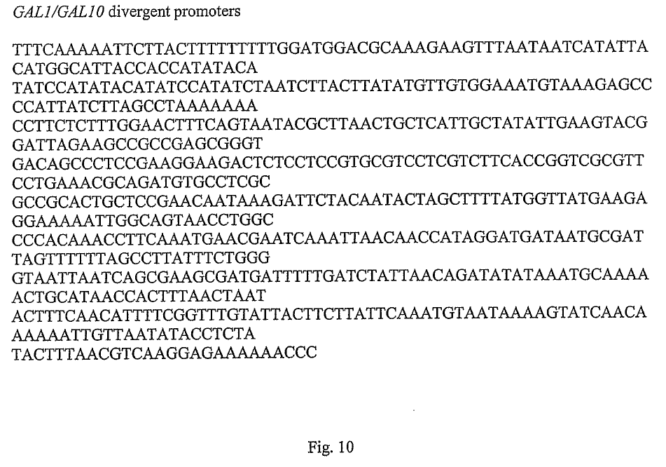

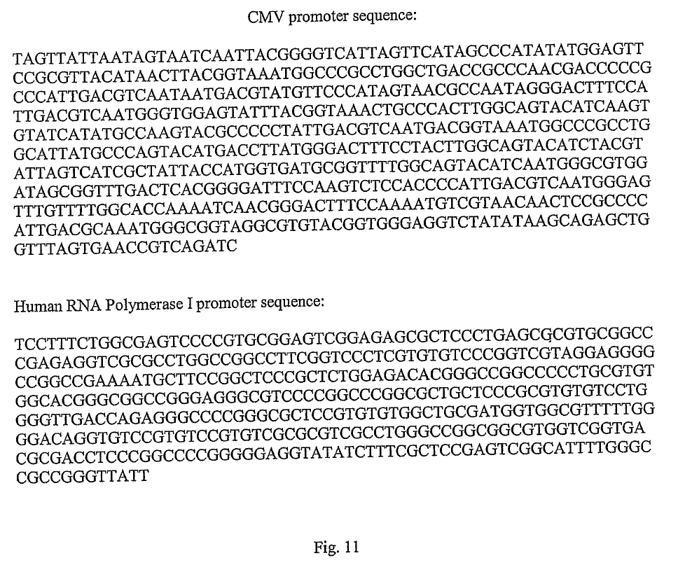

Key to the operation of group II introns are three short sequence elements that base pair with flanking 5′- and 3′-exon sequences to help position the splice junctions at the intron's active site for both RNA splicing and reverse splicing reactions (FIG. 6B) The sequence elements EBS1 and EBS2 (exon-binding sites 1 and 2) in DI each form 5 to 6 base pairs with the 5′-exon sequences IBS1 and IBS2 (intron-binding sites 1 and 2). As used herein, “IBS” refers to sequences in the target DNA substrate that lie immediately upstream of the targeted cleavage site. In group IIA introns, the sequence δ adjacent to EBS1 base pairs with δ′, typically the first 1-3 nucleotides of the 3′ exon, i.e., the first 1-3 nucleotides downstream from the target cleavage site, while in group 1113 introns, the 3′ exon base pairs instead with EBS3, located in a different part of DI (FIG. 6B).

FIG. 7: Reverse Transcriptase-Polymerase Chain Reaction (RT-PCR) to detect spliced exons from URA 3 directed targetron. Yeast were transformed with plasmid coexpressing URA3 targetron and LtrA protein and induced with galactose for the times indicated on the top of each lane. RNA was extracted and RTPCR with primers directed against URA3 exon sequences was performed. The arrows indicate size of the precursor RNA and ligated exons. Lane 1 has 1 Kb MW markers.

FIG. 8: Northern blot of intron RNA from yeast cells expressing intron RNA and LtrA protein. Lane 1, 1 Kb MW marker. Lane 2 & 3, in vitro prepared precursor and spliced RNA. Arrows indicate mobility of intron precursor and spliced lariat RNA. Lane 4, URA3 targetron intron casette in antisense orientation (negative control). Lanes 5-9, URA3 targetron intron in sense orientation. Lane 5 & 6, LtrA expressed with an NLS lane 7 without NLS. Lane 8 and 9 RNA and protein expressed in yeast strain XRN1⁻ lacking 5′ exonuclease. Lane 8 (no NLS on protein); lane 9 (no poly A signal on intron transcript).

FIG. 9 is a diagram depicting the nucleotide sequence of the aI2 intron RNA, SEQ.ID.NO. 1 and the nucleotide sequence of the group II intron RNA of the first intron of the S. cerevisiae mitochondrial COX1 gene, hereinafter referred to as the “aI1 intron” RNA, SEQ.ID.NO.2. Markings above the sequence identify the position of the EBS1 sequence and the EBS2 sequence of the wild-type all intron RNA and the wild-type aI2 intron RNA

### DETAILED DESCRIPTION OF THE INVENTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

### I. Definitions

“Group II intron DNA,” as used herein, is a specific type of DNA present in bacteria and in organelles, particularly the mitochondria of fungi, yeast and plants and the chloroplast of plants. The group II intron RNA molecules, that is, the RNA molecules which are encoded by the group II introns, share similar secondary and tertiary structures. The group II intron RNA molecules typically have six domains. Domain IV of the group II intron RNA contains the nucleotide sequence which encodes the “group II intron-encoded protein.”

“Excised group II intron RNA,” as used herein, refers to an RNA that is a transcript of the group II intron DNA that lacks flanking exon sequences.

“Group II intron encoded protein,” as used herein, is a protein encoded by an open reading frame within a group II intron. The group II intron-encoded protein comprises an X domain and a reverse transcriptase domain. The X domain of the protein is associated with maturase activity. In some cases, the proteins also comprise an En domain having a DNA endonuclease motif. As used herein, group II intron-encoded proteins also encompass modified group II intron-encoded proteins that have additional or altered amino acids at the N terminus, or C terminus, or alterations in the internal regions of the protein, as well as wild-type group II intron-encoded proteins.

“Modified,” as used herein, refers to DNA, RNA or proteins which differ from the wild-type form of the DNA, RNA, or protein. In the case of DNA or RNA, modified refers to one or more of substitutions, additions or deletions of nucleotides in the DNA or RNA sequence, such that the modified sequence is different from the normal, wild-type sequence. Modified can refer to substitutions, additions or deletions of nucleotides in a sequence within DNA or RNA that does not encode a protein, such as for example, one or more of the EBS1, EBS2 and δ regions of the group II intron. Modified can also refer to substitutions, additions or deletions of nucleotides, as compared to normal wild-type, within a protein-encoding sequence of the DNA or RNA. The protein encoded by such a modified protein-encoding DNA or RNA sequence could itself be modified in that it could have one or more of substitutions, additions or deletions of amino acids within its protein sequence as compared to the normal, wild-type sequence of the protein.

“DNA recognition sites,” as used herein, refer to the sequence of nucleotide bases within the DNA substrate which are recognized by the group II intron ribonucleprotein particles that are produced in accordance with the present methods, or components thereof, as signals to cleave the DNA substrate and then insert nucleic acid molecules into the substrate. DNA recognition sites can also be referred to as “targets” since these are sites into which nucleic acid molecules are inserted.

“DNA substrate,” as used herein, means the DNA molecule containing DNA recognition sites which are cleaved by the wild-type or modified group II intron ribonucleoprotein particles produced in accordance with the present methods and into which nucleic acid molecules are inserted.

“Promoter,” as used herein, refers to sequences in DNA which mediate initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements downstream of the transcription initiation site. The individual sequence elements function as sites on the DNA where RNA polymerases and transcription factors that facilitate positioning of RNA polymerases on the DNA bind.

“Flanking DNA”, as used herein, refers to a segment of DNA that is collinear with and adjacent to a particular point of reference

“Heterologous gene,” as used herein, refers to a nucleotide sequence, not normally encoded by a group II intron, that is inserted into a group II intron, preferably using recombinant DNA techniques. Such heterologous genes can then be inserted into the DNA substrate, at or near the DNA recognition site, as part of the process by which the group II intron encoding the heterologous gene, is inserted into the DNA substrate By way of example, the heterologous gene can comprise an entire open reading frame, one or more exons of a desirable gene, a promoter, a terminator, other cis acting regulatory elements or signal sequences.

“Localization signals,” as used herein, refer to amino acid or peptide sequences that are recognized intracellularly and selectively transported to specific locations within the cell. For example, localization signals exist, and are known in the art, that are responsible for transport to the nucleus, mitochondria and chloroplasts. By incorporating localization signals within other cellular proteins, it is possible to direct the entire protein to the intracellular location to which the specific peptide localization signal is transported. This can be done, preferably using recombinant DNA methodology, by fusing the DNA sequence encoding a specific localization signal to a gene encoding a protein that one wants to localize to a specific site in the cell.

Methods of enhancing production of group II intron RNP particles in eukaryotic cells are provided. In accordance with the present methods, at least one DNA construct encoding a wild-type or, preferably, a modified group II intron RNA and a wild-type or modified group II intron encoded protein, is introduced into the cell, and the cell maintained under conditions that allow expression of the group II intron RNA and the group II intron encoded protein. In certain embodiments, the group II intron RNA and the group II intron encoded protein may be operably linked to the same RNA polymerase promoter. In other embodiments, the group II intron RNA and the intron encoded protein are operably linked to different RNA polymerase promoters.

The group II intron RNP particles produced in accordance with the present methods comprise a wild type or modified excised group II intron and a wild-type or modified group II intron encoded protein. Such particles are capable of catalyzing the cleavage, at specific target sites, of DNA substrates in the cell and, in certain cases, causing the insertion of a heterologous gene into the target site. Because the DNA target site is recognized mainly by base pairing of the intron RNA, it is possible to develop group II intron ribonucleoprotein particles into gene targeting vectors (“targetrons”) to insert efficiently into any desired DNA target simply by modifying the group II intron RNA In addition to targeted gene disruption, the group II intron RNP particles can be used to introduce at desired chromosomal locations heterologous genes that have been cloned into domain IV of the excised group II intron RNA and to introduce targeted double-strand breaks that stimulate homologous recombination with a co-transformed DNA fragment, enabling the introduction of point mutations and/or nucleic acids of interest into the target site. (See, copending and commonly assigned PCT Application No. ______, which claims priority to U.S. Provisional Application 60/579,326, which was filed on Jun. 14, 2004.)

Reaction of the targeted DNA substrate with group II intron RNP particles in cells results initially in the insertion of the group II intron RNA molecule of the RNP particle into one strand of the double stranded DNA substrate at the cleavage site, then synthesis of a cDNA molecule which is complementary to the group II intron RNA molecule into the other strand of the double-stranded DNA substrate. Formation of this heteroduplex in the DNA target site occurs by a mechanism in which the excised group II intron RNA reverse splices directly into the DNA target site and is then reverse transcribed by the intron-encoded protein. Over time, this heteroduplex structure is converted to a double stranded DNA structure.

The group II intron RNP particles of the present invention are derived from group II introns. Wild-type group II introns are found in bacterial and organellar, primarily mitochondrial and chloroplast, genomes of lower eukaryotes and higher plants. They are also found in both gram-positive and gram negative bacteria, and a few archaea. The present application contemplates methods which produce group II RNP particles comprising sequences that encode wild-type and modified group II intron RNA and wild-type and modified group II intron encoded proteins derived from group IIA, group IIB, and group IIC introns in eukaryotic cells. Particularly good results have been achieved using RNP particles derived from bacterial group II introns such as the Lactococcal L1.LtrB group II intron of the Lactococcus lactis ltrB relaxase gene.

The RNP particles produced in accordance with the present methods comprise a group II intron-encoded protein which is bound to an excised group II intron RNA whose sequence is identical to a group II intron RNA that is found in nature, i.e., a wild-type group II intron RNA, or an excised group II intron RNA whose sequence is different from a group II intron RNA that is found in nature, i.e., a modified, excised group II intron RNA molecule. Modified excised group II intron RNA molecules, include, for example, group II intron RNA molecules that have nucleotide base changes or additional nucleotides in the internal loop regions of the group II intron RNA, preferably the internal loop region of domain IV, and group II intron RNA molecules that have nucleotide base changes in the hybridizing regions of domain I. RNP particles in which the group II intron RNA has nucleotide base changes in the hybridizing region, as compared to the wild type, typically have altered specificity for the DNA substrate of the wild-type RNP particle.

Targeting of the group II intron RNP particle involves base pairing of the excised modified or wild-type group II intron RNA of the RNP particle to a specific region of the DNA substrate. The group II intron RNA has two sequences, EBS1 and EBS2, that are capable of hybridizing with two intron RNA-binding sequences, IBS1 and IBS2, on one strand of the DNA substrate, hereinafter referred to as the “top” strand for convenience. Additional interactions occur between the intron-encoded protein and regions in the DNA substrate flanking the IBS1 and IBS2 sites. As denoted herein, nucleotides that are located upstream of the cleavage site have a (−) position relative to the cleavage site, and nucleotides that are located downstream of the cleavage site have a (+) position relative to the cleavage site. Thus, the cleavage site is located between nucleotides −1 and +1 on the top strand of the double-stranded DNA substrate. The IBS1 sequence and the IBS2 sequence lie in a region of the DNA substrate which extends from about position −1 to about position −14 relative to the cleavage site. Group IIA intron RNA molecules also comprise a sequence referred to as delta (δ) that base pairs with the nucleotides in the 3′ exon, typically +1 to +3 of the DNA substrate, a sequence that is referred to as δ′. Group IIB intron RNA molecules comprise a sequence referred to as EBS3 that base pairs with nucleotide residues in the 3′ exon of the targeted DNA substrate. The δ sequence is located in domain I of the group IIA intron RNA, while the EBS3 sequence is located in a different region of domain I of the group IIB intron RNA. (See FIG. 6)

EBS1 is located in domain I of the group II intron RNA and comprises from about 5 to 7 nucleotides that are capable of hybridizing to the nucleotides of the IBS1 sequence of the substrate. EBS2 is located in domain I of the group II intron RNA upstream of EBS1 and comprises from about 5 to 7 nucleotides that are capable of hybridizing to the nucleotides of IBS2 sequence of the substrate. In order to cleave the substrate efficiently, it is preferred that the δ sequence or the EBS3 sequence of the group II intron RNA, be complementary to nucleotides in the 3′ exon in the top strand of the substrate.

Examples of group II intron RNP particles which may be used in the present methods include, but are not limited to, the aI2 RNP particle, the all RNP particle, and the Lactococcal L1.LtrB intron RNP particles. The aI2 RNP particle comprises a wild-type or modified group II intron RNA of the second intron of the S. cerevisiae mitochondrial COX1 gene, hereinafter referred to as the “aI2 intron” RNA, bound to a wild-type or modified aI2 intron encoded-protein. EBS1 of the aI2 intron RNA comprises 6 nucleotides and is located at position 2985-2990 of the wild-type sequence. EBS1 of the wild-type aI2 intron RNA has the sequence 5′-AGAAGA. EBS2 of the aI2 intron RNA comprises 6 nucleotides and is located at positions 2935-2940. EBS2 of the wild-type aI2 intron RNA has the sequence 5′-UCAUUA.

The aI1 RNP particle comprises an excised, wild-type or modified group II intron RNA of the first intron of the S. cerevisiae mitochondrial COX1 gene, hereinafter referred to as the “aI1 intron” RNA, and a wild-type or modified aI1 intron-encoded protein. EBS1 of the aI1 intron RNA comprises 6 to 7 nucleotides and is located at position 426-431. EBS1 of the wild-type aI1 intron RNA has the sequence 5′-CGUUGA. EBS2 of the all intron RNA comprises 5 to 6 nucleotides and is located at positions 376-381. EBS2 of the wild-type aI1 intron RNA has the sequence 5′-ACAAUU.

The L1.LtrB intron RNP particle comprises an excised, wild-type or modified excised group II intron RNA of the Lactococcus lactis ltrB gene, hereinafter referred to as the “L1.LtrB intron” RNA, and a wild-type or modified L1.LtrB intron-encoded protein, hereinafter referred to as the LtrA protein. The sequence of the Lactococcal L1.LtrB intron is shown in the attached figure. The EBS1 of the Lactococcal L1.LtrB intron RNA comprises 7 nucleotides and is located at positions 457 to 463. The EBS1 sequence of the wild-type Lactococcal L1.LtrB intron RNA has the sequence 5′-GUUGUGG. The EBS2 of the Lactococcal L1.LtrB intron RNA comprises 6 nucleotides and is located at positions 401 to and including 406. The EBS2 sequence of the wild-type Lactococcal L1.ltrB intron RNA has the sequence 5′AUGUGU.

The modified RNP particle can catalyze the cleavage of DNA substrates and the insertion of nucleic acid molecules at new recognition sites in the DNA substrate. Because the recognition site of the DNA substrate is recognized, in part, through base pairing with the excised group II intron RNA of the functional RNP particle, it is possible to control the site of nucleic acid insertion within the DNA substrate. This is done by modifying the EBS1 sequence, the EBS2 sequence, the delta sequence, the EBS3 sequence or combinations thereof. Methods of modifying group II intron RNP particles such that they bind to and catalyze the cleavage of DNA substrates at different recognition sites are described in U.S. Pat. Nos. 5,698,421 and 6,027,895, both of which are incorporated herein by reference in their entirety.

The modified group II intron RNP particles are targeted to specific sites with the aid of a computer algorithm that scans the target sequence for the best matches to the positions recognized by the intron encoded parotein and then designs primers for modifying the base-pairing regions within the intron to insert into those sites (Perutka, J., Wang, W., Goerlitz, D., and Lambowitz, A. M. (2004) Use of computer-designed group II introns to disrupt Escherichia coli DexH/D-box protein and DNA helicase genes. J. Mol. Biol. 336, 421-439). The positions recognized by the intron-encoded proteins are sufficiently few and flexible that the algorithm readily identifies multiple rank-ordered target sites in any gene. Further, the intron can be targeted to insert in either strand, resulting in different orientations relative to the target gene. An intron that inserts in the antisense orientation gives an unconditional disruption, whereas an intron that integrates in the sense-orientation can potentially yield a conditional disruption by linking its splicing to the expression of the intron encoded protein from a separate construct with an inducible promoter (Karberg, M., Guo, H., Zhong, J., Coon, R., Perutka, J., and Lambowitz, A. M. (2001) Group II introns as controllable gene targeting vectors for genetic manipulation of bacteria. Nat. Biotechnol. 19, 1162-1167.; Frazier, C. L., San Filippo, J., Lambowitz, A. M., and Mills, D. A. (2003) Genetic manipulation of Lactococcus lactis by using targeted group II introns: generation of stable insertions without selection. Appl. Environ. Microbiol. 69, 1121-1128). Selectable markers are readily incorporated into the intron, but because integration frequencies are generally high, the desired integrants can often be identified by colony PCR screening even in the absence of selection (Perutka, J., Wang, W., Goerlitz, D., and Lambowitz, A. M. (2004) Use of computer-designed group II introns to disrupt Escherichia coli DexH/D-box protein and DNA helicase genes. J. Mol. Biol. 336, 421-439).

DNA molecules encoding modified group II intron RNA containing desired EBS sequences which hybridize to corresponding nucleotides on substrate DNA or containing additional nucleotides (e.g. a polynucleotide encoding a drug resistance marker) in domain IV may be prepared using standard genetic engineering procedures, such as in vitro site-directed mutagenesis.

Because the group II intron RNP particles of the present invention recognize their DNA target sites mainly by base pairing of the intron RNA, they can be targeted to insert into different DNA sites simply by modifying the intron RNA. This feature, combined with their very high insertion frequency and specificity, makes it possible to use the group II intron RNP particles of the present invention as programmable gene-targeting vectors in eukaryotic cells. Group II intron RNP particles can be used in eukaryotic cells for the site-specific chromosomal insertion of cargo genes cloned in domain IV of the RNA and to introduce targeted double-strand breaks, which stimulate homologous recombination with a co-transformed DNA fragment, enabling the introduction of point mutations.

### Constructs

The present invention provides constructs for enhancing the production of group II intron RNP particles in eukaryotic cells. In one embodiment, the construct comprises a nucleic acid encoding the group II intron RNA operably linked to an RNA polymerase I, an RNA polymerase II, or an RNA polymerase III promoter and a nucleic acid encoding the group II intron-encoded protein operably linked to an RNA polymerase II promoter, wherein the nucleic acid encoding the group II intron-encoded protein is downstream or upstream of the nucleic acid encoding the group II intron RNA.

Examples of suitable RNA polymerase I promoters include, but are not limited to, the human RNA polymerase I promoter and the mouse RNA polymerase I promoter. The sequences of species-specific RNA polymerase I promoters are known in the art. The sequence of the human polymerase I promoter is shown in the attached figure. Characteristics of the human RNA polymerase I promoter are described in Neumann G, Watanabe T, Ito H, Watanabe S, Goto H, Gao P, Hughes M, Perez D R, Donis R, Hoffmann E, Hobom G, Kawaoka Y. (1999) Generation of influenza A viruses entirely from cloned cDNAs. Proc. Natl. Acad. Sci. USA, 96, 9345-9350, which is specifically incorporated herein by reference. Preferably, the RNA polymerase I promoter is derived from the same species of animal as the cells into which the construct is introduced. The human RNA polymerase I promoter (Neumann et al., 1993) used in the examples below is minimal. Studies showed that the first 17 bp of rDNA transcript sequence were important for transcription efficiency (Smale S T, Tjian R. (1985) Transcription of herpes simplex virus tk sequences under the control of wild-type and mutant human RNA polymerase I promoters. Mol Cell Biol. 5, 352-62). The longer version of promoter (−500, +17) may be PCR amplified and used to replace the shorter version.

Examples of suitable RNA polymerase II promoters include, but are not limited to, the human cytomegalovirus (CMV) immediate early promoter, the thymidine kinase promoter, and the SV40 promoter. Examples of suitable RNA polymerase III promoters include, but are not limited to, the U6 snRNA promoter and H1 promoter (for the RNA component of RNase P)

In certain embodiments the sequence encoding the protein and the sequence encoding the group II intron RNA may both be operably linked to the same promoter, preferably a Pol II promoter. In those cases where the DNA encoding the group II intron RNA and the group II intron-encoded protein are operably linked to the same promoter, e.g., an RNA polymerase II promoter, and the sequence encoding the protein is downstream of the sequence encoding the group II intron RNA, the construct preferably also comprises an internal ribosome entry site (IRES) between the sequence encoding the group II intron RNA and the sequence encoding the protein. In other embodiments the sequence encoding the group II intron RNA and the sequence encoding the protein are operably linked to different promoters In such embodiments, the sequence encoding the modified group II intron RNA may be operably linked to an RNA polymerase I, II or III promoter, and the sequence encoding the group II intron encoded protein, preferably, is operably liked to an RNA polymerase II promoter.

The nucleic acid encoding the group II intron RNA, which may also be referred to as a “group II intron DNA sequence” for convenience, preferably lacks a sequence that encodes a portion of domain IV of the group II intron RNA, preferably from about 50% to about 90%, more preferably from about 65% to about 90%, most preferably from about 80% to about 90% of the loop region of domain IV, while retaining a plurality of nucleotides at the 5′ end and the 3′ end of domain IV. Preferably, about 95 to about 200 nucleotides are retained at the 5′ end and about 25 to about 150 nucleotides are retained the 3′ end of domain IV. As a result of the deletion, the group II intron DNA sequence does not encode a full-length protein. Depending upon the intron and the size of the deletion, the group II intron DNA sequence either comprises no open reading frame or a disrupted open reading frame which encodes a truncated protein. In certain embodiments, a heterologous gene is incorporated into domain IV of the group II intron RNA. In those cases where the heterologous gene comprises a sequence encoding a protein, peptide, or a desirable RNA, a promoter, either a constitutive or, preferably, an inducible promoter, is operably linked to the protein, peptide or RNA coding sequence. In other embodiments, the heterologous sequence is a promoter. Alternatively the heterologous gene comprises an IRES followed by the protein, peptide or RNA encoding sequence. The heterologous gene is any sequence.

In those cases where the group II intron DNA sequence and the sequence encoding the group II intron-encoded protein are in the same construct, the protein-encoding sequence, preferably, is located either upstream or downstream of the group II intron sequence. Thus, the construct can contain a single promoter which drives transcription of the group II intron RNA and expression of the protein. Alternatively, the construct can contain two promoters, one of which drives transcription of the group II intron RNA, and one of which drives expression of the protein. Preferably, the construct further comprises sequences which flank the group II intron DNA sequence and allow splicing of the group II intron RNA from the intron transcript. Such sequences are complementary to the EBS1, EBS2, and δ or EBS3 sequences of the group II intron RNA. Optionally, the constructs of the present application are incorporated into a plasmid that contains an origin of replication to allow for amplification of the construct.

In another embodiment, the construct of the present invention comprises a sequence encoding the group II intron encoded protein and lacking a sequence that encodes the group II intron RNA, i.e., the sequences encoding the group II intron RNA and the group II intron encoded protein are incorporated into different constructs. In such embodiments, it is preferred that the construct containing the protein encoding sequence comprise an RNA polymerase II promoter operably linked to the protein encoding sequence.

In certain embodiments, the constructs of the present invention also comprise a nucleic acid encoding a nuclear localization signal (referred to hereinafter as an “NLS”) linked to the 5′ end or, preferably, the 3′ end of the protein encoding sequence. One example of such NLS is the SV40 NLS. The characteristics of other suitable nuclear localization sequences are described in Jans, D. A. Protein transport to the nucleus and its regulation. In ‘Protein Targeting’, IRL press, Oxford, edited by Hurtley, S. M., Science International, ICRL Press, pp25-62. Alternatively, the constructs of the present invention may comprise a nucleic acid encoding a nucleolar localization sequence linked to the 5′ end or the 3′ end of the sequence encoding the group II intron encoded protein.

In those cases where the sequence encoding the group II intron encoded protein is in the same construct and downstream of the sequence encoding the group II intron RNA, the construct, preferably, also comprises an internal ribosome entry site (IRES) and an in frame ATG codon between the 3′ end of the sequence encoding the group II intron RNA and the sequence encoding the protein. In certain embodiments, the construct comprising the protein coding sequence also contains a spliceosomal intron.

In certain embodiments of the present constructs, the sequence encoding the group II intron RNA is not linked to a polyadenylation signal while the sequence encoding the protein is linked to a polyadenylation signal.

In certain embodiments, the intron-encoded protein sequences in the present constructs contain codons that are recognized and preferred by the translational regulatory molecules of a eukaryotic cell, more particularly an animal cell, such as a human cell.

### Methods of Introducing the Nucleic Acid Constructs Into the Eukaryotic Cells

In another aspect, the present invention provides methods which use the constructs of the present invention to enhance production of functional group II intron RNP particles in eukaryotic cells. The nucleic acid constructs of the present invention are introduced into the host eukaryotic cell by cloning the construct into a vector and by introducing the vector into the host cell by conventional methods, such as electroporation, lipid-based or calcium phosphate-mediated transfection procedures. The method used to introduce the DNA molecule is related to the particular host cell used. To be introduced into eukaryotic cells, the DNA sequence is preferably inserted into viral or other vectors, such as for example, an SV40-derived expression vector, an adenovirus-derived expression vector, an adeno-associated virus vector, a poxvirus-derived viral vector, Herpes-simplex virus-derived vectors, Vaccinia virus vectors, Vesicular Stomatitis virus vectors, Measles virus vectors, or plasmid vectors. In those instances where the host cell has different codon usages from the protein-coding sequence to be introduced into the cell, the protein coding sequence of the construct may be modified to comprise codons that are optimal for the host cell. The protein coding sequence, typically, is modified by using a DNA synthesizer or by in vitro site directed mutagenesis to prepare an open reading frame sequence with preferred codons. Alternatively, to resolve the differences in the codon usage of the protein encoding sequence and that of the host cell, sequences that encode the tRNA molecules which correspond to the optimal codons of the protein encoding sequences are introduced into the host cell. Optionally, DNA molecules which comprise sequences that encode factors that assist in RNA or protein folding, or that inhibit RNA or protein degradation are also introduced into the cell.

In one embodiment two constructs are introduced into the eukaryotic host cell, one of which contains the group II intron RNA encoding sequence and one of which contains the protein-encoding sequence. In another embodiment, a single construct that comprises both the group II intron RNA encoding sequence and protein encoding sequence are introduced into the host cells. Following introduction of the DNA molecule into the eukaryotic cell, the group II intron DNA sequence is transcribed into intron RNA precursor. The intron then excises itself out from the precursor with the help of the protein expressed from the intron-encoded ORF. The excised intron and the intron-encoded protein stay bound as RNP particles. Optionally, magnesium ions are also introduced into the cells to increase production of the functional RNP particles. (See, copending and commonly assigned PCT Application No. ______, which claims priority to U.S. Provisional Application 60/579,326, which was filed on Jun. 14, 2004.)

The following examples are included for purposes of illustration and are not intended to limit the scope of the invention.

### EXAMPLES

### Example 1

### Expression of Lactococcus lactis L1.L1trB intron Intron-Encoded Maturase LtrA in Mammalian Cells

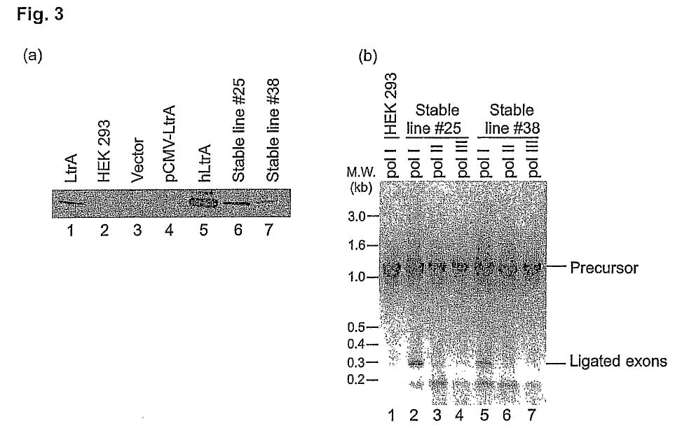

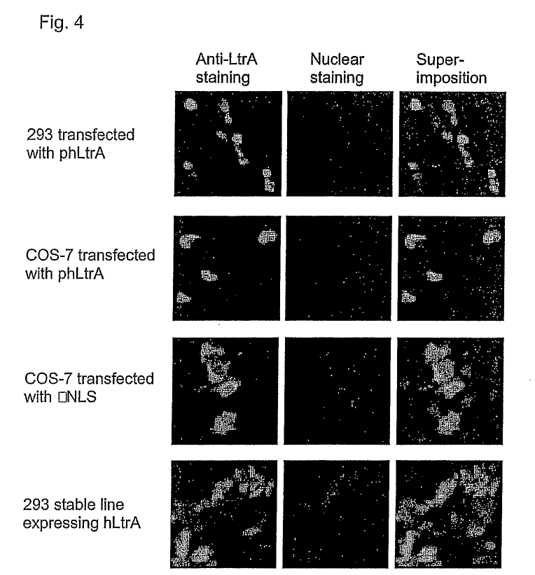

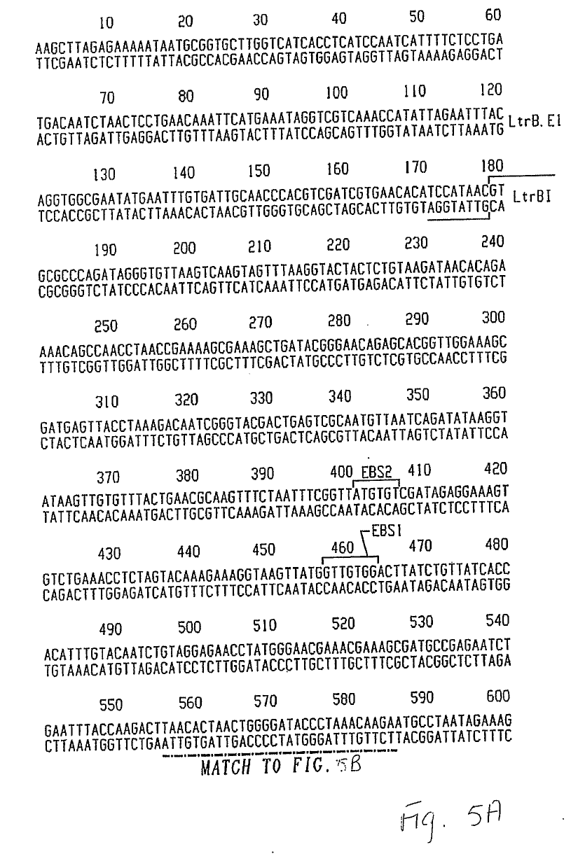

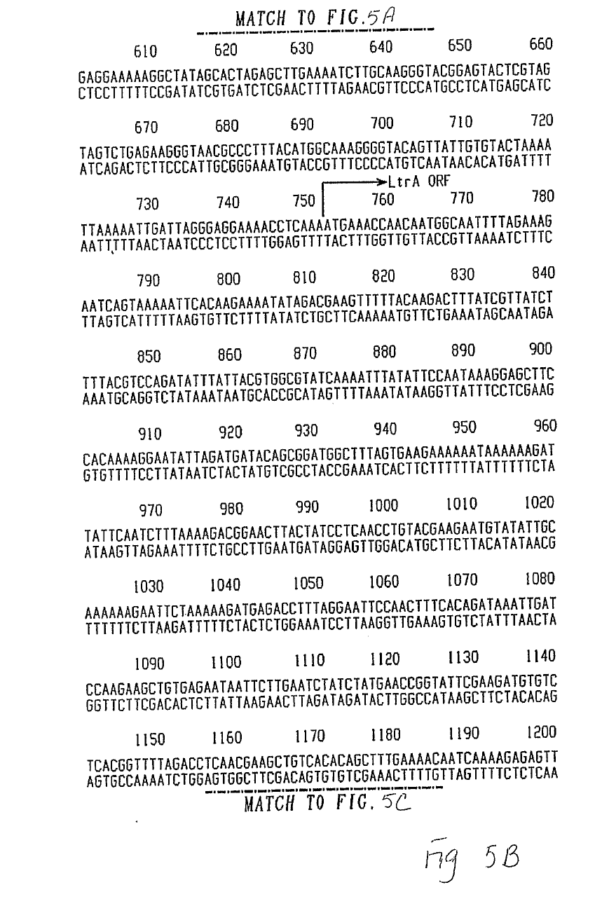

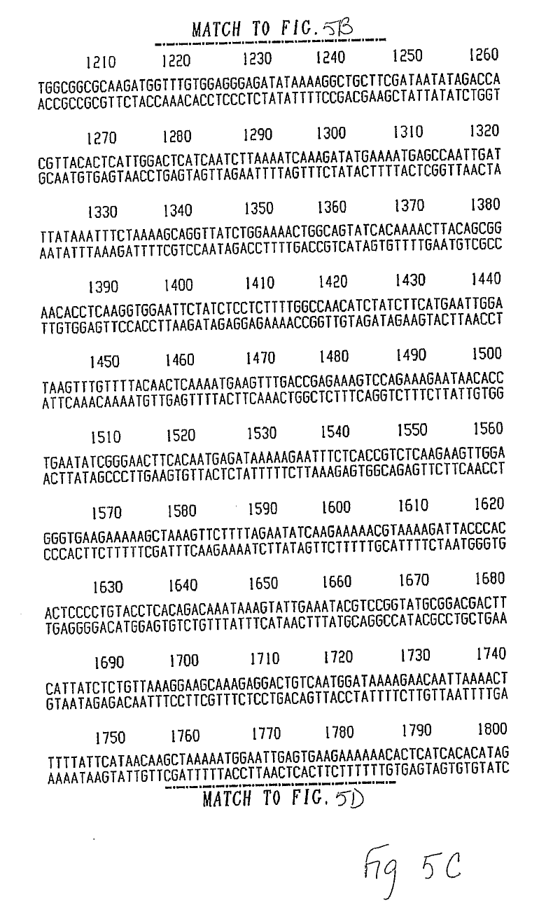

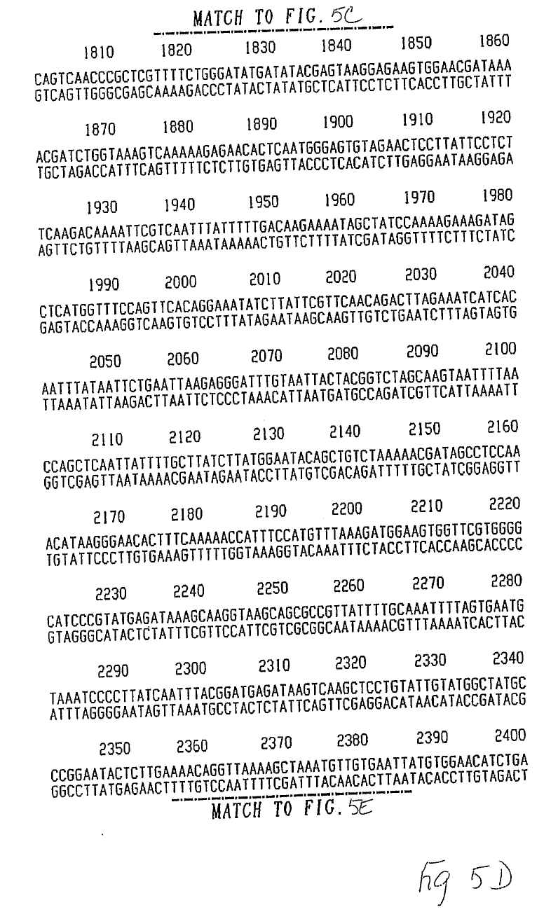

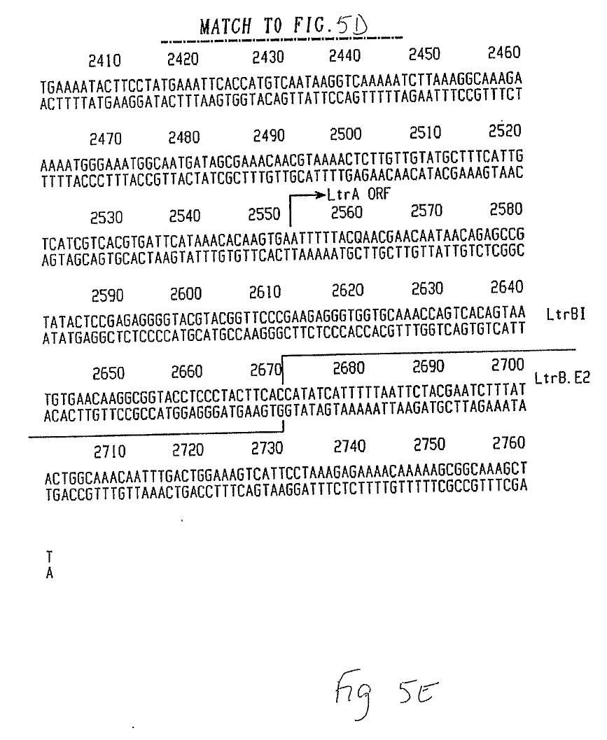

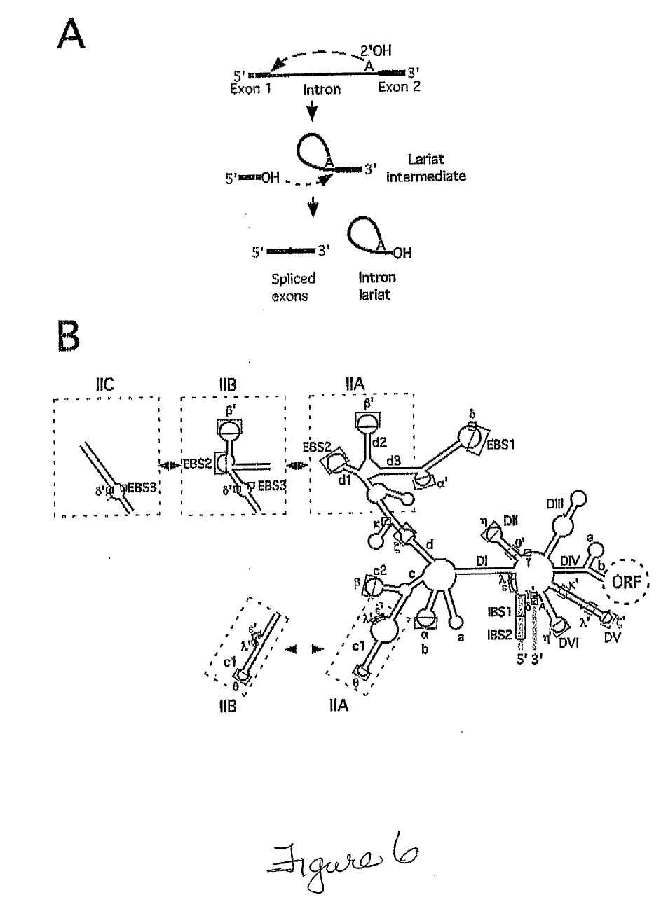

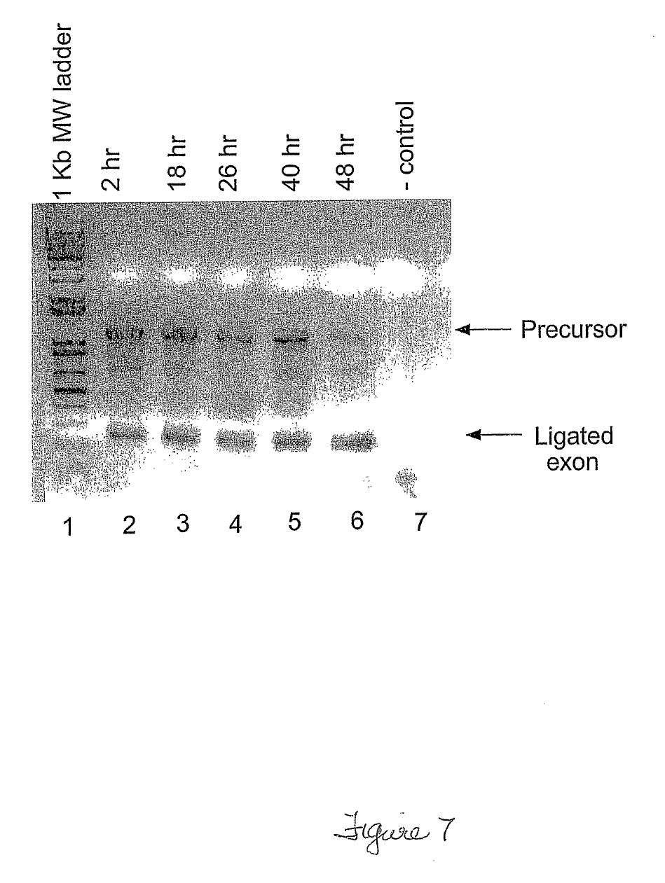

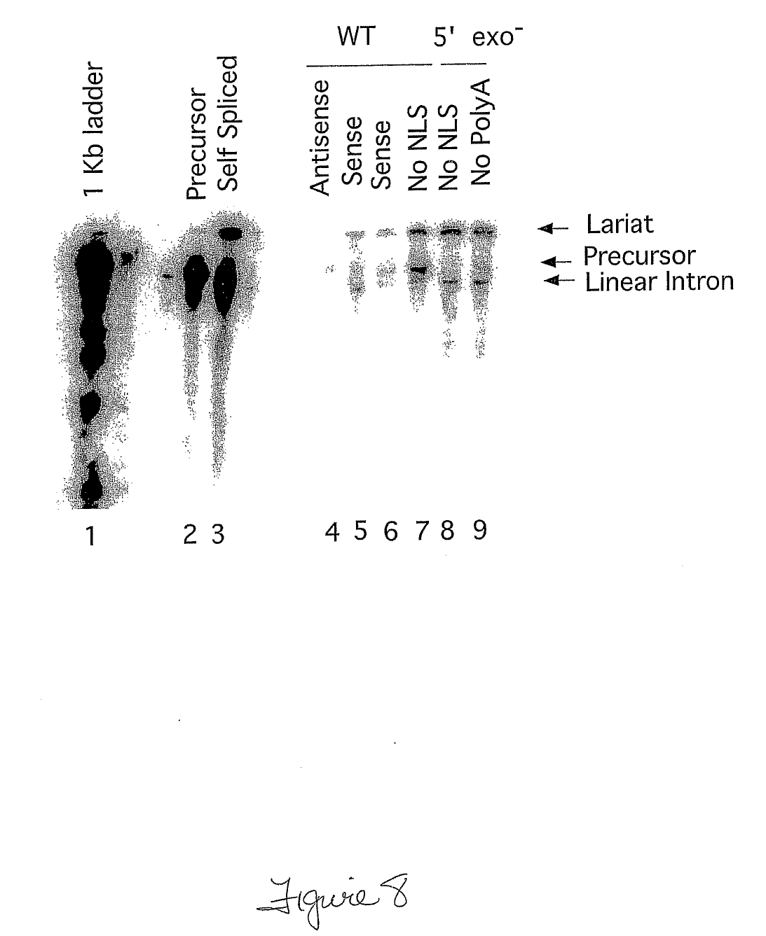

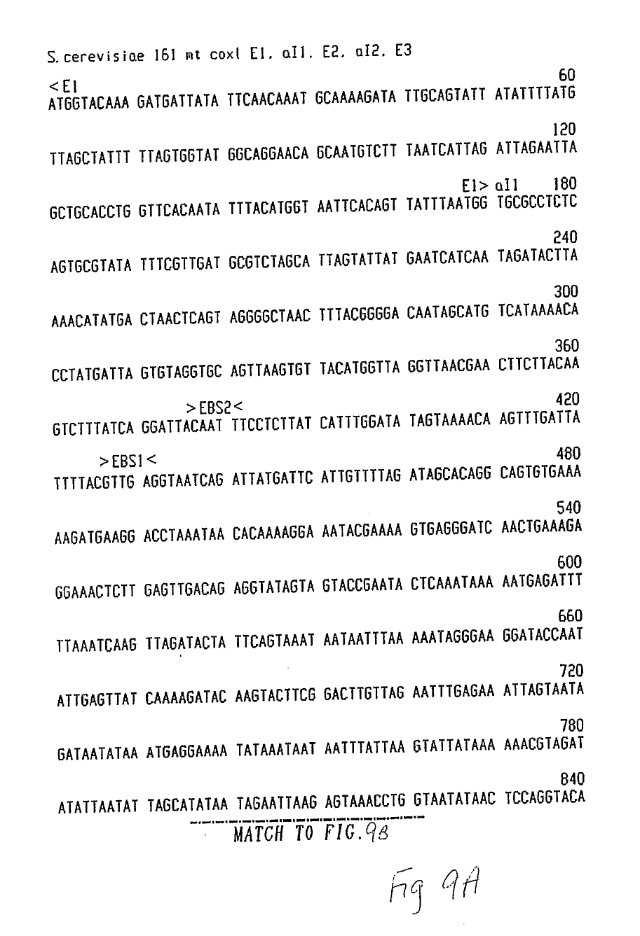

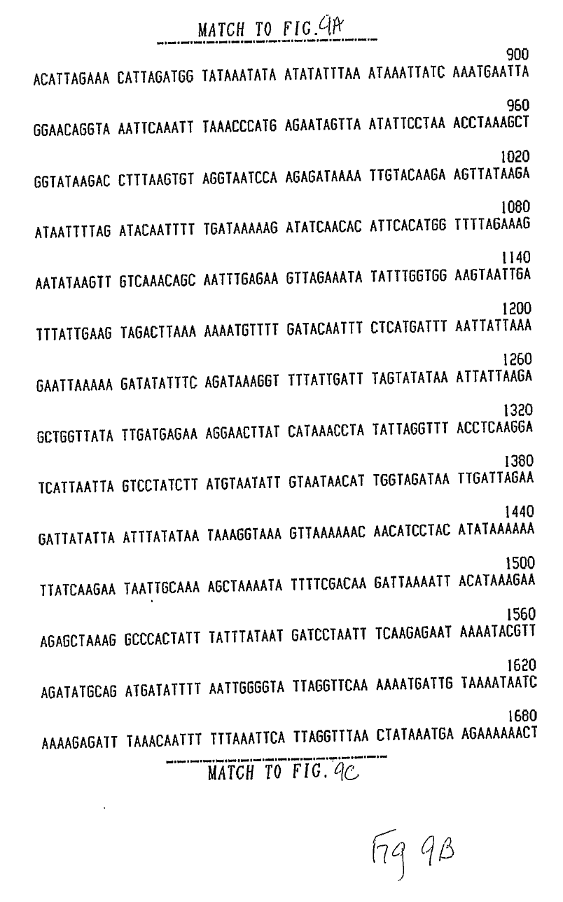

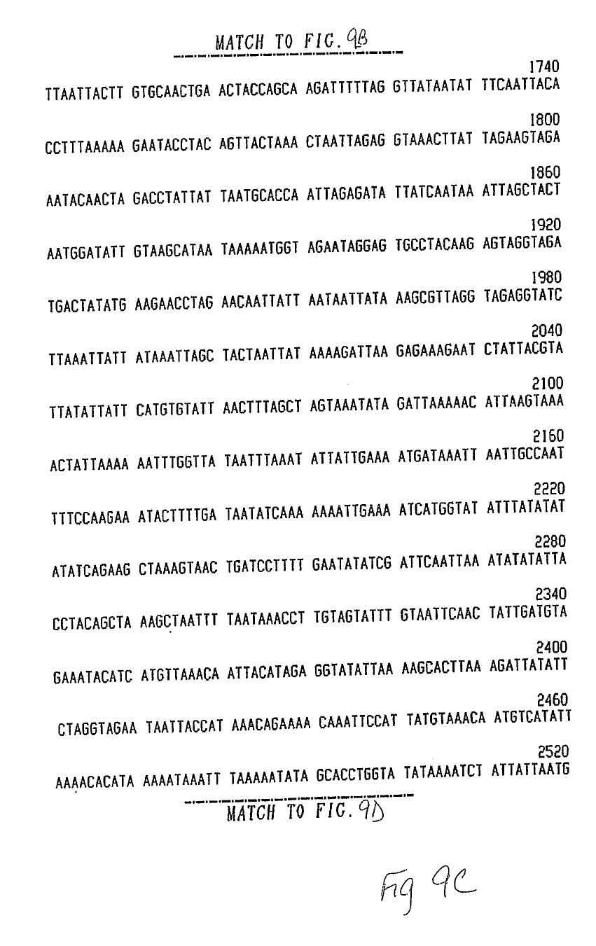

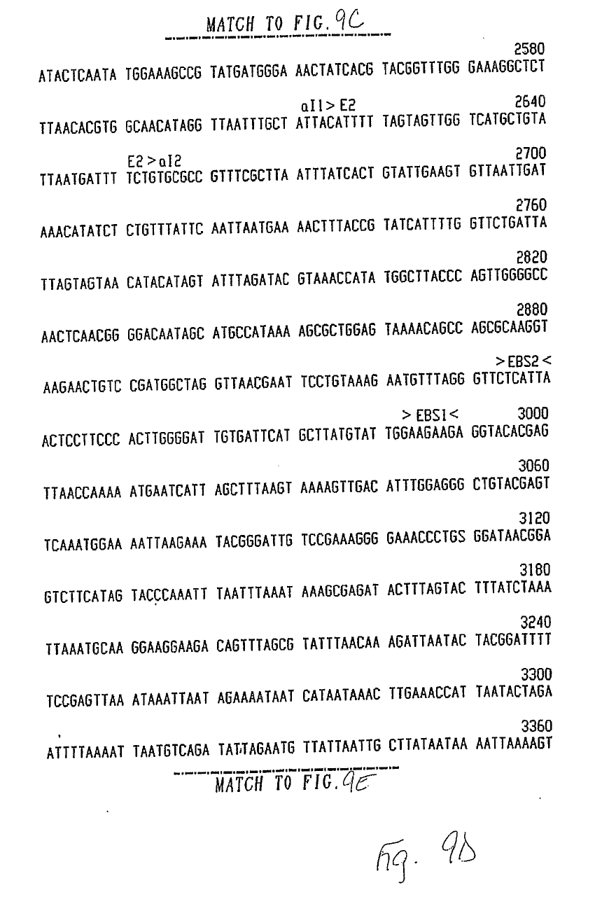

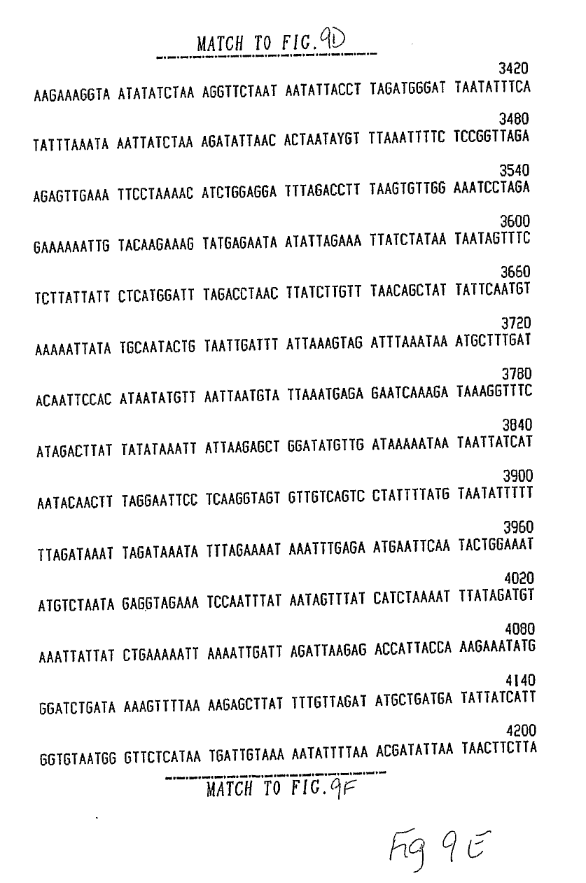

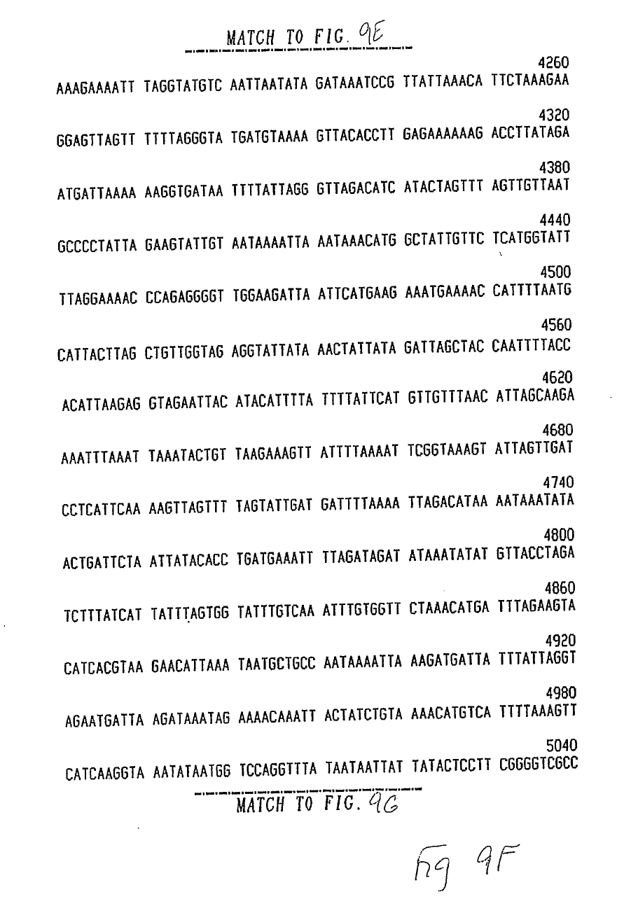

The LtrA open reading frame was first cloned into the pCMV/myc/nuc plasmid between NcoI and XhoI sites, with a spliceable 133 bp IVS sequence (presence of a conventional spliceosomal intron allows for optimal protein expression in eukaryotic cells) inserted between the CMV promoter (an RNA polymerase II promoter) and the start codon to promote expression (Le Hir, H., Nott, A., and Moore, M. J. (2003) How introns influence and enhance eukaryotic gene expression. Trends in Biochemistry Sciences, 28, 215-220). When transfected with pCMV/myc/nuc-LtrA, neither HeLa nor COS-7 cells showed LtrA expression detected by anti-LtrA antibody or anti-myc antibody in Western analysis (FIG. 3a and not shown).

We noticed that most codons of LtrA are highly unfavorable to mammals, which could cause inefficient translation of the mRNA and may also form spliceosomal recognition sites and lead to mRNA truncation. Codon optimization was a potential solution.

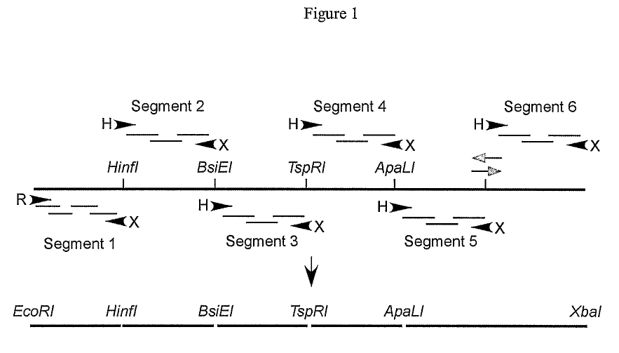

Codon usage was based on Haas, J., Park, E., and Seed, B. (1996) Codon usage limitation in the expression of HIV-1 envelope glycoprotein. Current Biol. 6, 315-324. Codon-optimized LtrA sequence was divided into several segments, each of which was flanked by two restriction sites that do not cut the particular fragment. Primers of 98- to 120-base long were synthesized and used as overlapping templates and PCR amplified by short primers of 30-45 bases. The PCR products were then cloned into pBluescriptKS vector for sequencing. The complete LtrA sequence was obtained by ligating all the segments after redigestion with the appropriate enzymes (see FIG. 1).

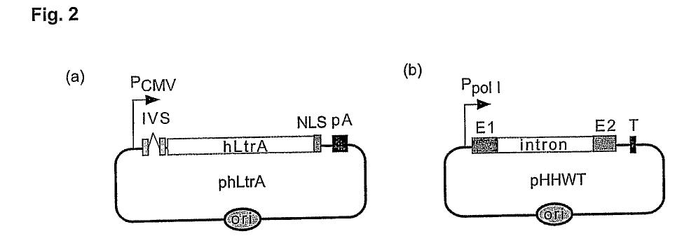

Expression vector phLtrA was constructed by cloning the LtrA open reading frame with the SV40 nuclear localization signal (NLS) at the C-terminus to vector pIRES (Clontech) between the EcoRI and NotI sites, so that the humanized LtrA gene (hLtrA) is preceded by a CMV promoter and the spliceable IVS mentioned above and followed by an SV40 polyA signal (FIG. 2a).

When transfected into HEK 293 and COS-7 cell lines, hLtrA expressed well as shown in Western analysis in FIG. 3a (lane 5), compared to no expression with untransfected cells (lane 2), vector without LtrA gene (lane 3), and the bacterial LtrA construct (lane 4). We also successfully made HEK 293 lines stably expressing hLtrA under a CMV promoter, indicating that overexpression of LtrA at a moderate level is not toxic to the cells (lanes 6 and 7).

### Example 2

### Expression of the intron RNA in Mammalian Cells

To express the intron RNA, we tested three different types of promoters, human RNA polymerase I promoter (FIG. 2b), CMV promoter—a RNA polymerase II promoter, and U6 promoter—a RNA polymerase III promoter. When transfected alone or co-transfected with phLtrA, intron precursor RNA was detected using RT-PCR with all the samples. However, ligated exon, indicative of splicing, was only observed in cells expressing both the Pol I construct (PHHWT) and phLtrA (FIG. 3b). The correct exon junction was confirmed by sequencing.

### Example 3

### Expression of RNP Particles in Yeast

### Yeast Plasmids Construction

A computer program (Perutka et. al) was used to select sites for intron insertion in the URA3 gene of Saccharomyces cerevisiae. Introns were designed to insert between positions 528/529 and 543/544 on the sense strand and 221/222 on the antisense strand (insertion sites numbered relative to the ATG in the coding sequence of URA3/Yel021W on chromosome V from coordinates 116167 to 116970 SGDID=S0000000747, GENEID:856692). The desired changes in EBS/IBS were engineered via PCR using the strategy outlined in Perutka et. al. Oligonucleotides used were
U528IBS(5′AAAAAAGCTTATAATTATCCTTAAAGAGCGACAAAGTGCGCCCAGAT AGGGTG), SEQ ID NO:
U528EBS1(5′CAGATTGTACAAATGTGGTGATAACAGATAAGTCGACAAAGATAAC TTACCTTTCTTTGT), SEQ ID NO:
EBS2 (5′TGAACGCAAGTTTCTAATTTCGATTCTCTTTCGATAGAGGAAAGTGTCT), SEQ ID NO:
ASEBS2 (5′CGAAATTAGAAACTTGCGTTCAGTAAAC), SEQ ID NO:

Engineered mutant introns were confirmed by sequencing and tested for mobility frequency as described in Perutka et.al.

The yeast expression vector pESC-Leu (Invitrogen, Carlsbad, Calif.) was used to express intron RNA and LtrA protein from a divergent galactose promoter. The LtrA protein coding sequence was amplified via PCR. The 5′ primer YEAST5 introduces a BamH1 site, SV40 NLS for nuclear targeting and reads
5′CG GGA TCC GCC ACC ATG GGT GCT CCT CCA AAA AAG AAG AGA AAG GTT GCT GGT ATC AAT AAA GAC ATC CCT GGT ATGAAACCAACAATGGCA, SEQ ID NO:—and the 3′ primer YEASTLT3
(5′CAATGATCATTACTTGTGTTTATGAATCACGTG), SEQ ID NO: introduces a BclI site. The PCR product was cleaved with BamHI and BclI and cloned into the BamHI site of pEsc-Leu. Clones with the insert under control of the GAL1 promoter were sequenced and one clone pLtrA51EscLeu was retained. The intron donor was amplified from the constructs above via PCR using primers 5SACLTRB
(5′-ATGCGAGCTCGGAATTGTGAGCGGATAACAATTCCCCTC), SEQ ID NO: and 3SACLTRB
(5′-CGAACGAGCTCTTCTTAAAGTTAAACAAAATTATTTCTAG), SEQ ID NO:. The PCR product was cleaved with SacI and cloned into the SacI site of pLtrA51EscLeu. Intron sequence was verified and clones with the intron expressed under control of the Gal10 promoter were retained.

### Yeast Transformation

Yeast expression plasmids were transformed into the desired strain using a high efficiency transformation protocol (Gietz R D, Woods R A. (2002) Transformation of yeast by lithium acetate/single-stranded carrier DNA/polyethylene glycol method. Methods Enzymol. 350:87-96). Transformants were selected on minimal plates supplemented appropriately for strain auxotrophies while allowing selection for the desired plasmid. Transformants were restreaked on minimal plates and maintained under selection.

### Galactose Induction

Freshly restreaked transformants were grown in 5 ml minimal media supplemented appropriately using 1% raffinose as the carbon source. The culture was diluted into 50 ml of the same media and 2% galactose was added at an OD₆₀₀ of ˜0.5. Samples were withdrawn at 3-24 hours and approximately 10⁸ cells were plated on FOA plates and the equivalent amount reserved for RNA preparations.

Yeast RNA preparation

RNA was extracted using Yeast RNA extraction Kit (Ambion). RNA was fractionated on 5% denaturing polyacrylamide gels and electroblotted onto nylon membrane and cross-linked to the membrane via a Stratalinker. The membranes were probed with an oligonucleotide designed to hybridize to the intron and labeled with ³²p. RTPCR was performed using standard conditions.

The URA3 gene in yeast encodes orotidine-5-phosphate decarboxylase. 5-fluoroorotic acid (FOA) is metabolized to 5-fluorouracil by the decarboxylase. The 5-fluorouracil can form fluorodeoxyuridine which inhibits thymidine synthase and is thus toxic to cells. ura3 cells can be selected on media containing containing FOA (Boeke J D, Trueheart J, Natsoulis G, Fink G R. (1987) 5-Fluoroorotic acid as a selective agent in yeast molecular genetics. Methods Enzymol. 154:164-75). The spontaneous mutation rate to ura3 is ˜3.3×10⁸. Designing group II intron targetrons to URA3 combined with FOA selection offers a strong selection for intron insertion. Towards this end group II introns for insertion into URA3 were designed via computer (Perutka et al) and tested in E. coli. An intron designed to insert in the sense strand at position U528 had a mobility frequency of 40% in E. coli and was transferred to a yeast shuttle vector pESC-LEU under an inducible galactose promoter. The vector pESC-Leu (Invitrogen) allows expression of LtrA protein and URA3 targeted intron RNA to be expressed from divergent GAL promoters. The LtrA protein has an SV-40 NLS appended to the N-terminus to allow the protein to be targeted to the nucleus. The transcript for both intron RNA and LtrA protein have polyadenylation signals on the 3′ end and thus are capable of being polyadenylated and exported from the nucleus. The galactose promoter is a pol II promoter that is normally repressed when cells are grown in presence of a sugar such as glucose or raffinose. On addition of galactose the promoter is rapidly induced on raffinose grown cells and transcripts expressing intron precursor and LtrA protein are produced. To determine if the intron precursor is spliced by the LtrA protein expressed RT PCR was performed. FIG. 7 shows a PCR product consistent with spliced exons is detectable. Direct sequencing of these products shows that the PCR product does contain spliced exons and splicing is accurate. FIG. 8 shows a northern showing presence of intron lariat (lanes 5-9). Together this demonstrates that the two essential components of a targetron (intron lariat and active LtrA protein) are being produced in this system. In other constructs the polyadenylation signal for the intron expressing casette was deleted, thus trapping the intron RNA in the nucleus. The northern blot (FIG. 8, lane 9) shows spliced intron lariat can be detected demonstrating that splicing can be detected in transcripts restricted to the nucleus. Spliced intron lariat is also detected in the presence of a polyadenylation signal for the intron expressing casette but in the absence of an NLS on the LtrA protein (lanes 7, 8, FIG. 8) demonstrating that active RNP's can be formed in the cytoplasm. The RNP's can be formed in a variety of nuclear backgrounds that have desirable properties that might influence the stability or activity of the RNP. These include, but are not restricted to mutants in 5′ and 3′ exonucleases, components of the exosome, chromatin remodelling enzymes, non sense mediated decay and debranching enzyme alone or in combinations. FIG. 8 (lanes 8, 9), show the influence of reconstituting RNP's in one such nuclear background. Strains mutant in XRN1 are deficient in a 5′ to 3′ exonuclease involved in RNA decay. FIG. 8 lanes 8 and 9 show presence of spliced lariat intron and stabilization of linear spliced intron.

What is claimed is:

1. A method for enhancing production in a eukaryotic cell of RNP particles comprising a modified or wild-type group II intron RNA associated with a modified or wild-type group II intron encoded-protein, comprising:
a) introducing into the eukaryotic cell a construct comprising:
i) a nucleic acid encoding a modified or wild-type group II intron RNA, wherein the nucleic acid encoding the group II intron RNA is operably linked to an RNA polymerase I promoter, an RNA polymerase II promoter, or an RNA polymerase III promoter; wherein the nucleic acid encoding the modified group II intron RNA lacks at least a portion of the group II intron open reading frame sequence, and
ii) a nucleic acid encoding a modified or wild-type group II intron-encoded protein, wherein the nucleic acid encoding the group II intron encoded protein is operably linked to an RNA polymerase II promoter and wherein the nucleic acid encoding the group II intron-encoded protein is upstream or downstream of the nucleic acid encoding the group II intron RNA, and
b) maintaining the eukaryotic cell under conditions that allow for expression of the group II intron RNA and the group II intron-encoded protein in the eukaryotic cell.

2. The method of claim 1, wherein the nucleic acid encoding the group II intron encoded protein is downstream of the nucleic acid encoding the group II intron RNA, wherein the nucleic acid encoding the group II intron encoded protein and the group II intron RNA are both linked to the same promoter, and wherein the construct comprises an internal ribosome entry site between the nucleic acid encoding the group II RNA and the nucleic acid encoding the group II intron encoded protein.

3. The method of claim 1 wherein the nucleic acid encoding the group II intron encoded protein is operably linked to an RNA polymerase II promoter, and the nucleic acid encoding the group II intron RNA is operably linked to an RNA polymerase I promoter or an RNA polymerase III promoter.

4. The method of any one of claims 1-3, wherein the group II intron RNA is a wild-type or modified bacterial group II intron RNA and wherein the group II intron encoded protein is encoded by a wild-type or modified bacterial group II intron.

5. The method of claim 4, wherein the group II intron RNA is a modified bacterial group II intron RNA.

6. The method of any one of claims 1-3, wherein the group II intron RNA is a modified L1.LtrB intron RNA, and wherein the group II intron encoded protein is encoded by the open reading frame of a wild-type or modified L1.LtrB intron.

7. The method of any one of claims 1-3 wherein the nucleic acid encoding the group II intron encoded protein is linked to a nuclear localization signal.

8. The method of any one of claims 1-3, wherein the codons encoding the group II intron encoded protein are modified to use codons that are preferred in eukaryotic cells.

9. A method for enhancing production in a eukaryotic cell of RNP particles comprising a modified or wild-type group II intron RNA associated with a modified or wild-type group II intron encoded-protein, comprising:
a) introducing into the eukaryotic cell
i.) a construct comprising a nucleic acid encoding a modified group II intron RNA, wherein the nucleic acid encoding the group II intron RNA is operably linked to an RNA polymerase I promoter, an RNA polymerase II promoter, or an RNA polymerase III promoter; and wherein the nucleic acid encoding the modified group II intron RNA lacks at least a portion of the group II intron RNA open reading frame sequence, and
ii) a construct comprising a nucleic acid encoding a modified or wild-type group II intron-encoded protein, wherein the nucleic acid encoding the group II intron encoded protein is operably linked to an RNA polymerase II promoter, and
b) maintaining the eukaryotic cell under conditions that allow for expression of the group II intron RNA and the group II intron-encoded protein in the eukaryotic cell.

10. The method of claim 9, wherein the nucleic acid encoding the group II intron RNA is operably linked to an RNA polymerase I promoter or an RNA polymerase III promoter.

11. The method of claim 9, wherein the group II intron RNA is a modified bacterial group II intron and wherein the group II intron encoded protein is encoded by a bacterial group II intron.

12. The method of claim any one of claim 9-11, wherein the group II intron RNA is encoded by a wild-type or modified lactoccocal L1.LtrB intron, and wherein the group II intron encoded protein is encoded by the open reading frame of a wild-type or modified lactoccocal L1.LtrB intron.

13. The method of any one of claims 9-12, wherein the nucleic acid encoding the group II intron encoded protein is linked to a nuclear localization signal.

14. The method of any one of claims 9-13, wherein the codons encoding the group II intron encoded protein are modified to use codons that are preferred in eukaryotic cells.

15. A construct comprising:
i) a nucleic acid encoding a modified or wild-type group II intron RNA, wherein the nucleic acid encoding the group II intron RNA is operably linked to an RNA polymerase I promoter, an RNA polymerase II promoter, or an RNA polymerase III promoter; wherein the nucleic acid encoding the modified group II intron RNA lacks at least a portion of the group II intron open reading frame sequence, and
ii) a nucleic acid encoding a modified or wild-type group II intron-encoded protein, wherein the nucleic acid encoding the group II intron encoded protein is operably linked to an RNA polymerase II promoter and wherein the nucleic acid encoding the group II intron-encoded protein is upstream or downstream of the nucleic acid encoding the group II intron RNA.

16. The construct of claim 15, wherein the nucleic acid encoding the group II intron RNA and the group II intron-encoded protein are operably linked to the same RNA polymerase promoter,
wherein the nucleic acid encoding the group II intron encoded protein is downstream of the nucleic acid encoding the group II RNA, and
wherein the construct comprises an internal ribosome entry site between the nucleic acid encoding the group II RNA and the nucleic acid encoding the group II intron encoded protein.

17. The construct of claim 15, wherein the nucleic acid encoding the group II intron RNA and the nucleic acid encoding the group II intron encoded protein are operably linked to different promoters.

18. The construct of claim 17 wherein the nucleic acid encoding the group II intron encoded protein is operably linked to an RNA polymerase II promoter.

19. The construct of any one of claims 15-18, wherein the construct comprises a subcellular localization signal encoding sequence operably linked to the sequence encoding the group II intron-encoded protein.

20. The construct of claim 19, wherein the subcellular localization signal encoding sequence is a nuclear or nucleolar localization signal encoding sequence.

21. The construct of claim 19, wherein the subcellular localization signal encoding sequence is at the 5′ end or 3′ end of the nucleic acid encoding the group II intron encoded protein.

22. The construct of any one of claims 15-18, wherein the nucleic acid encoding the group II intron encoded protein comprises codons preferred by human cells.

23. The construct of any one of claim 15-22, wherein the construct comprises a splicesomal intron, and wherein the splicesomal intron is located between the promoter and the sequence encoding the group II intron encoded protein.

24. The construct of any one of claim 15-23, wherein sequences that allow for splicing of the group II intron RNA from the transcript of the nucleic acid encoding the group II intron RNA are attached to the 5′ end and the 3′ end of the sequence encoding the group II intron RNA.

25. A construct comprising a nucleic acid sequence encoding a wild-type or modified group II intron encoded protein operably linked to an RNA polymerase II promoter.

26. The construct of claim 25, comprising a splicesomal intron, wherein the splicesomal intron is located between the promoter and the sequence encoding the group II intron encoded protein.

27. The construct of claim 25, comprising a polyadenylation sequence downstream of the nucleic acid sequence encoding the group II intron encoded protein.

28. The construct of any one of claims 25-27, wherein said construct lacks a nucleic acid sequence encoding a group II intron RNA.

29. A construct comprising a nucleic acid encoding a modified group II intron RNA operably linked to an RNA polymerase I promoter.

30. The construct of claim 29, wherein the construct lacks a sequence encoding a group II intron encoded protein.


SEQUENCE LISTING (16 sequences)

SEQ ID NO: 1 - DNA, 2523 bp, Saccharomyces cerevisiae
gtgcgccgtttcgcttaatttatcactgtattgaagtgttaattgataaacatatctctgtttattcaattaatgaaaactttaccgtatcattttggttctgattattagtagtaacatacatagtatttagatacgtaaaccatatggcttacccagttggggccaactcaacggggacaatagcatgccataaaagcgctggagtaaaacagccagcgcaaggtaagaactgtccgatggctaggttaacgaattcctgtaaagaatgtttagggttctcattaactccttcccacttggggattgtgattcatgcttatgtattggaagaagaggtacacgagttaaccaaaaatgaatcattagctttaagtaaaagttgacatttggagggctgtacgagttcaaatggaaaattaagaaatacgggattgtccgaaaggggaaaccctgsggataacggagtcttcatagtacccaaatttaatttaaataaagcgagatactttagtactttatctaaattaaatgcaaggaaggaagacagtttagcgtatttaacaaagattaatactacggatttttccgagttaaataaattaatagaaaataatcataataaacttgaaaccattaatactagaattttaaaattaatgtcagatattagaatgttattaattgcttataataaaattaaaagtaagaaaggtaatatatctaaaggttctaataatattaccttagatgggattaatatttcatatttaaataaattatctaaagatattaacactaataygtttaaattttctccggttagaagagttgaaattcctaaaacatctggaggatttagacctttaagtgttggaaatcctagagaaaaaattgtacaagaaagtatgagaataatattagaaattatctataataatagtttctcttattattctcatggatttagacctaacttatcttgtttaacagctattattcaatgtaaaaattatatgcaatactgtaattgatttattaaagtagatttaaataaatgctttgatacaattccacataatatgttaattaatgtattaaatgagagaatcaaagataaaggtttcatagacttattatataaattattaagagctggatatgttgataaaaataataattatcataatacaactttaggaattcctcaaggtagtgttgtcagtcctattttatgtaatatttttttagataaattagataaatatttagaaaataaatttgagaatgaattcaatactggaaatatgtctaatagaggtagaaatccaatttataatagtttatcatctaaaatttatagatgtaaattattatctgaaaaattaaaattgattagattaagagaccattaccaaagaaatatgggatctgataaaagttttaaaagagcttattttgttagatatgctgatgatattatcattggtgtaatgggttctcataatgattgtaaaaatattttaaacgatattaataacttcttaaaagaaaatttaggtatgtcaattaatatagataaatccgttattaaacattctaaagaaggagttagttttttagggtatgatgtaaaagttacaccttgagaaaaaagaccttatagaatgattaaaaaaggtgataattttattagggttagacatcatactagtttagttgttaatgcccctattagaagtattgtaataaaattaaataaacatggctattgttctcatggtattttaggaaaacccagaggggttggaagattaattcatgaagaaatgaaaaccattttaatgcattacttagctgttggtagaggtattataaactattatagattagctaccaattttaccacattaagaggtagaattacatacattttattttattcatgttgtttaacattagcaagaaaatttaaattaaatactgttaagaaagttattttaaaattcggtaaagtattagttgatcctcattcaaaagttagttttagtattgatgattttaaaattagacataaaataaatataactgattctaattatacacctgatgaaattttagatagatataaatatatgttacctagatctttatcattatttagtggtatttgtcaaatttgtggttctaaacatgatttagaagtacatcacgtaagaacattaaataatgctgccaataaaattaaagatgattatttattaggtagaatgattaagataaatagaaaacaaattactatctgtaaaacatgtcattttaaagttcatcaaggtaaatataatggtccaggtttataataattattatactccttcggggtcgccgcgggggcgggccggactattaaatatgcgttaaatggagagccgtatgatatgaaagtatcacgtacggttcggagagggctcttttatatgaatgttattacattcagataggtttgctactctactcttagta

SEQ ID NO: 2 - DNA, 2448 bp, Saccharomyces cerevisiae
gtgcgcctctcagtgcgtatatttcgttgatgcgtctagcattagtattatgaatcatcaatagatacttaaaacatatgactaactcagtaggggctaactttacggggacaatagcatgtcataaaacacctatgattagtgtaggtgcagttaagtgttacatggttaggttaacgaacttcttacaagtctttatcaggattacaatttcctcttatcatttggatatagtaaaacaagtttgattattttacgttgaggtaatcagattatgattcattgttttagatagcacaggcagtgtgaaaaagatgaaggacctaaataacacaaaaggaaatacgaaaagtgagggatcaactgaaagaggaaactcttgagttgacagaggtatagtagtaccgaatactcaaataaaaatgagatttttaaatcaagttagatactattcagtaaataataatttaaaaatagggaaggataccaatattgagttatcaaaagatacaagtacttcggacttgttagaatttgagaaattagtaatagataatataaatgaggaaaatataaataataatttattaagtattataaaaaacgtagatatattaatattagcatataatagaattaagagtaaacctggtaatataactccaggtacaacattagaaacattagatggtataaatataatatatttaaataaattatcaaatgaattaggaacaggtaaattcaaatttaaacccatgagaatagttaatattcctaaacctaaagctggtataagacctttaagtgtaggtaatccaagagataaaattgtacaagaagttataagaataattttagatacaatttttgataaaaagatatcaacacattcacatggttttagaaagaatataagttgtcaaacagcaatttgagaagttagaaatatatttggtggaagtaattgatttattgaagtagacttaaaaaaatgttttgatacaatttctcatgatttaattattaaagaattaaaaagatatatttcagataaaggttttattgatttagtatataaattattaagagctggttatattgatgagaaaggaacttatcataaacctatattaggtttacctcaaggatcattaattagtcctatcttatgtaatattgtaataacattggtagataattgattagaagattatattaatttatataataaaggtaaagttaaaaaacaacatcctacatataaaaaattatcaagaataattgcaaaagctaaaatattttcgacaagattaaaattacataaagaaagagctaaaggcccactatttatttataatgatcctaatttcaagagaataaaatacgttagatatgcagatgatattttaattggggtattaggttcaaaaaatgattgtaaaataatcaaaagagatttaaacaattttttaaattcattaggtttaactataaatgaagaaaaaactttaattacttgtgcaactgaactaccagcaagatttttaggttataatatttcaattacacctttaaaaagaatacctacagttactaaactaattagaggtaaacttattagaagtagaaatacaactagacctattattaatgcaccaattagagatattatcaataaattagctactaatggatattgtaagcataataaaaatggtagaataggagtgcctacaagagtaggtagatgactatatgaagaacctagaacaattattaataattataaagcgttaggtagaggtatcttaaattattataaattagctactaattataaaagattaagagaaagaatctattacgtattatattattcatgtgtattaactttagctagtaaatatagattaaaaacattaagtaaaactattaaaaaatttggttataatttaaatattattgaaaatgataaattaattgccaattttccaagaaatacttttgataatatcaaaaaaattgaaaatcatggtatatttatatatatatcagaagctaaagtaactgatccttttgaatatatcgattcaattaaatatatattacctacagctaaagctaattttaataaaccttgtagtatttgtaattcaactattgatgtagaaatacatcatgttaaacaattacatagaggtatattaaaagcacttaaagattatattctaggtagaataattaccataaacagaaaacaaattccattatgtaaacaatgtcatattaaaacacataaaaataaatttaaaaatatagcacctggtatataaaatctattattaatgatactcaatatggaaagccgtatgatgggaaactatcacgtacggtttgggaaaggctctttaacacgtggcaacataggttaatttgctattacat

SEQ ID NO: 3 - DNA, 5203 bp, Saccharomyces cerevisiae
atggtacaaagatgattatattcaacaaatgcaaaagatattgcagtattatattttatgttagctatttttagtggtatggcaggaacagcaatgtctttaatcattagattagaattagctgcacctggttcacaatatttacatggtaattcacagttatttaatggtgcgcctctcagtgcgtatatttcgttgatgcgtctagcattagtattatgaatcatcaatagatacttaaaacatatgactaactcagtaggggctaactttacggggacaatagcatgtcataaaacacctatgattagtgtaggtgcagttaagtgttacatggttaggttaacgaacttcttacaagtctttatcaggattacaatttcctcttatcatttggatatagtaaaacaagtttgattattttacgttgaggtaatcagattatgattcattgttttagatagcacaggcagtgtgaaaaagatgaaggacctaaataacacaaaaggaaatacgaaaagtgagggatcaactgaaagaggaaactcttgagttgacagaggtatagtagtaccgaatactcaaataaaaatgagatttttaaatcaagttagatactattcagtaaataataatttaaaaatagggaaggataccaatattgagttatcaaaagatacaagtacttcggacttgttagaatttgagaaattagtaatagataatataaatgaggaaaatataaataataatttattaagtattataaaaaacgtagatatattaatattagcatataatagaattaagagtaaacctggtaatataactccaggtacaacattagaaacattagatggtataaatataatatatttaaataaattatcaaatgaattaggaacaggtaaattcaaatttaaacccatgagaatagttaatattcctaaacctaaagctggtataagacctttaagtgtaggtaatccaagagataaaattgtacaagaagttataagaataattttagatacaatttttgataaaaagatatcaacacattcacatggttttagaaagaatataagttgtcaaacagcaatttgagaagttagaaatatatttggtggaagtaattgatttattgaagtagacttaaaaaaatgttttgatacaatttctcatgatttaattattaaagaattaaaaagatatatttcagataaaggttttattgatttagtatataaattattaagagctggttatattgatgagaaaggaacttatcataaacctatattaggtttacctcaaggatcattaattagtcctatcttatgtaatattgtaataacattggtagataattgattagaagattatattaatttatataataaaggtaaagttaaaaaacaacatcctacatataaaaaattatcaagaataattgcaaaagctaaaatattttcgacaagattaaaattacataaagaaagagctaaaggcccactatttatttataatgatcctaatttcaagagaataaaatacgttagatatgcagatgatattttaattggggtattaggttcaaaaaatgattgtaaaataatcaaaagagatttaaacaattttttaaattcattaggtttaactataaatgaagaaaaaactttaattacttgtgcaactgaactaccagcaagatttttaggttataatatttcaattacacctttaaaaagaatacctacagttactaaactaattagaggtaaacttattagaagtagaaatacaactagacctattattaatgcaccaattagagatattatcaataaattagctactaatggatattgtaagcataataaaaatggtagaataggagtgcctacaagagtaggtagatgactatatgaagaacctagaacaattattaataattataaagcgttaggtagaggtatcttaaattattataaattagctactaattataaaagattaagagaaagaatctattacgtattatattattcatgtgtattaactttagctagtaaatatagattaaaaacattaagtaaaactattaaaaaatttggttataatttaaatattattgaaaatgataaattaattgccaattttccaagaaatacttttgataatatcaaaaaaattgaaaatcatggtatatttatatatatatcagaagctaaagtaactgatccttttgaatatatcgattcaattaaatatatattacctacagctaaagctaattttaataaaccttgtagtatttgtaattcaactattgatgtagaaatacatcatgttaaacaattacatagaggtatattaaaagcacttaaagattatattctaggtagaataattaccataaacagaaaacaaattccattatgtaaacaatgtcatattaaaacacataaaaataaatttaaaaatatagcacctggtatataaaatctattattaatgatactcaatatggaaagccgtatgatgggaaactatcacgtacggtttgggaaaggctctttaacacgtggcaacataggttaatttgctattacatttttagtagttggtcatgctgtattaatgattttctgtgcgccgtttcgcttaatttatcactgtattgaagtgttaattgataaacatatctctgtttattcaattaatgaaaactttaccgtatcattttggttctgattattagtagtaacatacatagtatttagatacgtaaaccatatggcttacccagttggggccaactcaacggggacaatagcatgccataaaagcgctggagtaaaacagccagcgcaaggtaagaactgtccgatggctaggttaacgaattcctgtaaagaatgtttagggttctcattaactccttcccacttggggattgtgattcatgcttatgtattggaagaagaggtacacgagttaaccaaaaatgaatcattagctttaagtaaaagttgacatttggagggctgtacgagttcaaatggaaaattaagaaatacgggattgtccgaaaggggaaaccctgsggataacggagtcttcatagtacccaaatttaatttaaataaagcgagatactttagtactttatctaaattaaatgcaaggaaggaagacagtttagcgtatttaacaaagattaatactacggatttttccgagttaaataaattaatagaaaataatcataataaacttgaaaccattaatactagaattttaaaattaatgtcagatattagaatgttattaattgcttataataaaattaaaagtaagaaaggtaatatatctaaaggttctaataatattaccttagatgggattaatatttcatatttaaataaattatctaaagatattaacactaataygtttaaattttctccggttagaagagttgaaattcctaaaacatctggaggatttagacctttaagtgttggaaatcctagagaaaaaattgtacaagaaagtatgagaataatattagaaattatctataataatagtttctcttattattctcatggatttagacctaacttatcttgtttaacagctattattcaatgtaaaaattatatgcaatactgtaattgatttattaaagtagatttaaataaatgctttgatacaattccacataatatgttaattaatgtattaaatgagagaatcaaagataaaggtttcatagacttattatataaattattaagagctggatatgttgataaaaataataattatcataatacaactttaggaattcctcaaggtagtgttgtcagtcctattttatgtaatatttttttagataaattagataaatatttagaaaataaatttgagaatgaattcaatactggaaatatgtctaatagaggtagaaatccaatttataatagtttatcatctaaaatttatagatgtaaattattatctgaaaaattaaaattgattagattaagagaccattaccaaagaaatatgggatctgataaaagttttaaaagagcttattttgttagatatgctgatgatattatcattggtgtaatgggttctcataatgattgtaaaaatattttaaacgatattaataacttcttaaaagaaaatttaggtatgtcaattaatatagataaatccgttattaaacattctaaagaaggagttagttttttagggtatgatgtaaaagttacaccttgagaaaaaagaccttatagaatgattaaaaaaggtgataattttattagggttagacatcatactagtttagttgttaatgcccctattagaagtattgtaataaaattaaataaacatggctattgttctcatggtattttaggaaaacccagaggggttggaagattaattcatgaagaaatgaaaaccattttaatgcattacttagctgttggtagaggtattataaactattatagattagctaccaattttaccacattaagaggtagaattacatacattttattttattcatgttgtttaacattagcaagaaaatttaaattaaatactgttaagaaagttattttaaaattcggtaaagtattagttgatcctcattcaaaagttagttttagtattgatgattttaaaattagacataaaataaatataactgattctaattatacacctgatgaaattttagatagatataaatatatgttacctagatctttatcattatttagtggtatttgtcaaatttgtggttctaaacatgatttagaagtacatcacgtaagaacattaaataatgctgccaataaaattaaagatgattatttattaggtagaatgattaagataaatagaaaacaaattactatctgtaaaacatgtcattttaaagttcatcaaggtaaatataatggtccaggtttataataattattatactccttcggggtcgccgcgggggcgggccggactattaaatatgcgttaaatggagagccgtatgatatgaaagtatcacgtacggttcggagagggctcttttatatgaatgttattacattcagataggtttgctactctactcttagtaatgcctgctttaattggaggttttggt

SEQ ID NO: 4 - DNA, 667 bp, Saccharomyces cerevisiae
tttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccc

SEQ ID NO: 5 - DNA, 2761 bp, Lactobacillus lactis
aagcttagagaaaaataatgcggtgcttggtcatcacctcatccaatcattttctcctgatgacaatctaactcctgaacaaattcatgaaataggtcgtcaaaccatattagaatttacaggtggcgaatatgaatttgtgattgcaacccacgtcgatcgtgaacacatccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggtaagttatggttgtggacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggaacttcacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagaagtggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaattaagagggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtggggcatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtggaacatctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgaatttttacgaacgaacaataacagagccgtatactccgagaggggtacgtacggttcccgaagagggtggtgcaaaccagtcacagtaatgtgaacaaggcggtacctccctacttcaccatatcatttttaattctacgaatctttatactggcaaacaatttgactggaaagtcattcctaaagagaaaacaaaaagcggcaaagctt

SEQ ID NO: 6 - DNA, 1800 bp, Lactobacillus lactis
atgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggaacttcacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagaagtggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaattaagagggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtggggcatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtggaacatctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtga

SEQ ID NO: 7 - DNA, 589 bp, Artificial Sequence
  Description of Artificial Sequence Synthetic CMV promoter sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 8 - DNA, 409 bp, Homo sapiens
tcctttctggcgagtccccgtgcggagtcggagagcgctccctgagcgcgtgcggcccgagaggtcgcgcctggccggccttcggtccctcgtgtgtcccggtcgtaggaggggccggccgaaaatgcttccggctcccgctctggagacacgggccggccccctgcgtgtggcacgggcggccgggagggcgtccccggcccggcgctgctcccgcgtgtgtcctggggttgaccagagggccccgggcgctccgtgtgtggctgcgatggtggcgtttttggggacaggtgtccgtgtccgtgtcgcgcgtcgcctgggccggcggcgtggtcggtgacgcgacctcccggccccgggggaggtatatctttcgctccgagtcggcattttgggccgccgggttatt

SEQ ID NO: 9 - DNA, 53 bp, Artificial Sequence
  Description of Artificial Sequence Synthetic oligonucleotide
aaaaaagcttataattatccttaaagagcgacaaagtgcgcccagatagggtg

SEQ ID NO: 10 - DNA, 60 bp, Artificial Sequence
  Description of Artificial Sequence Synthetic oligonucleotide
cagattgtacaaatgtggtgataacagataagtcgacaaagataacttacctttctttgt

SEQ ID NO: 11 - DNA, 49 bp, Artificial Sequence
  Description of Artificial Sequence Synthetic oligonucleotide
tgaacgcaagtttctaatttcgattctctttcgatagaggaaagtgtct

SEQ ID NO: 12 - DNA, 28 bp, Artificial Sequence
  Description of Artificial Sequence Synthetic oligonucleotide
cgaaattagaaacttgcgttcagtaaac

SEQ ID NO: 13 - DNA, 92 bp, Artificial Sequence
  Description of Artificial Sequence Synthetic primer
cgggatccgccaccatgggtgctcctccaaaaaagaagagaaaggttgctggtatcaataaagacatccctggtatgaaaccaacaatggca

SEQ ID NO: 14 - DNA, 33 bp, Artificial Sequence
  Description of Artificial Sequence Synthetic primer
caatgatcattacttgtgtttatgaatcacgtg

SEQ ID NO: 15 - DNA, 39 bp, Artificial Sequence
  Description of Artificial Sequence Synthetic primer
atgcgagctcggaattgtgagcggataacaattcccctc

SEQ ID NO: 16 - DNA, 40 bp, Artificial Sequence
  Description of Artificial Sequence Synthetic primer
cgaacgagctcttcttaaagttaaacaaaattatttctag